(19) United States
(12) Patent Application Publication
(10) Pub. No.: US 2007/0168136 A1
(43) Pub. Date: Jul. 19, 2007

(54) SYSTEM AND METHOD FOR ASSESSING DIABETIC CONDITIONS

(76) Inventors: ROBERT C. BOOTH, COLUMBUS, NC (US); RICHARD L. GROUNSELL, GREENVILLE, SC (US); MAURICE E. DURSCHLAG, CHARLOTTE, NC (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/625,131

(22) Filed: Jan. 19, 2007

Related U.S. Application Data
(63) Continuation-in-part of application No. 11/529,224, filed on Sep. 29, 2006.
(60) Provisional application No. 60/760,156, filed on Jan. 19, 2006.

(51) Int. Cl. G06F 19/00; A61B 5/00

(52) U.S. Cl. 702/19; 600/300

(57) ABSTRACT

A system is provided for a keto-acidosis protocol which includes a computer processor having patient parameter data input. The patient parameter data input to the computer processor may include the patient's blood glucose level, a target patient blood glucose level, a patient's carbon dioxide level, a patient's sodium level, a patient's blood pH value, and the patient's potassium level which has been measured. A patient hydration computer program assesses the patient's hydration state. Coupled to the patient hydration computer program is a patient potassium computer program to assess the patient's potassium level and a patient blood pH value computer program assesses the patient's blood pH value. A display monitor is coupled to the computer processor for displaying indicia thereon responsive to the actuation of the patient hydration computer program, the patient potassium computer program, and the patient blood pH value computer program.

### CROSS-REFERENCE TO RELATED APPLICATIONS

This application is a Continuation-in-Part Application of U.S. patent application Ser. No. 11/529,224, filed on 29 Sep. 2006 and is based on U.S. Provisional Patent Application Ser. No. 60/760,156, filed on 19 Jan. 2006.

### BACKGROUND OF THE INVENTION

The maintenance of blood glucose levels of patients within a preferred target range is an important criteria to the physical well being of a patient. In the determination of possible diabetic conditions for a patient, the criteria of potassium levels, pH blood levels, and the hydration characteristic of the patient is of importance in the management of these associated conditions.

A number of factors are associated with glucose levels of a patient external to a target range including genetic abnormality, trauma due to injury, conditions arising from surgical procedures, as well as a number of other physical factors including potassium levels of the patient, serum pH values, and the hydration status of the patient.

High blood glucose levels are defined as hyperglycemia which occurs when a patient's blood glucose level is above a preferred target range. Hyperglycemia is caused by having excess glucose and/or not enough insulin in the patient's body. Symptoms of diabetes are the same as the symptoms for hyperglycemia where diabetes itself may cause the hyperglycemia. Furthermore, hyperglycemia may lead to other diabetic conditions, which may include keto-acidosis.

Hypoglycemia is defined as being a condition where the patient's blood glucose is below a preferred target range and is caused by not having enough glucose in the body to bring the patient's blood level into the preferred target range.

The subject invention concept is directed to an automated system which assesses diabetic conditions of a patient and which includes the assessment of a patient's hydration state, potassium level, and pH blood value to be used in conjunction with other parameters to provide an overall system and method for measuring as well as predicting insulin dosing rates.

Management of a patient's blood glucose level is important in diabetic patients where blood glucose levels are outside of a preferred target range and may cause serious health complications, including blindness, kidney failure, heart disease and extremity amputations.

Different types of diabetes may be treated in a number of manners and may differ between the particular type of diabetes which affects a particular patient. Dosing rates depending on the type of diabetes vary in bringing a patient's blood glucose level into the preferred target range.

Type 1 diabetes is generally and commonly referred to as an insulin-dependent diabetes mellitus or juvenile-onset diabetes which is developed when the body's immune system destroys pancreatic beta cells which make hormone insulin that regulates blood glucose.

Type 2 diabetes may be commonly referred to as a non-insulin dependent diabetes mellitus or adult-onset diabetes. This type of diabetes may be generally initiated as insulin resistance where the cells do not properly use the insulin provided by the body.

### FIELD OF THE INVENTION

The subject system and method is directed to both a system and method for measuring and predicting optimal insulin dosing rates in order to bring a patient's blood glucose level into a preferred target range.

The subject concept is further directed to a system having a computer-directed formula system for evaluation of current as well as cumulative patient blood glucose values based upon the aggregate of the measurements computed by the computer system. Calculation is provided and recommended insulin dosing rates are predicted to drive the blood glucose level of the patient into a preferred and predetermined target range.

In particular, it has been found that the hydration state of the patient, the potassium level of the patient, and the serum pH of the patient is of importance in responding to diabetic conditions resulting from hyperglycemia, such as, keto-acidosis.

The subject system directs itself to a portable system where an attending physician and/or caregiver may be provided with an alarm or other type of warning to be alerted to the fact that the patient's blood glucose level is external to the preferred target range.

Additionally, the subject system and method relates to both a system and method whereby information derived from the calculated blood-glucose dosing rate may be transmitted automatically to an external station which may be through a wireless transmission or a hard linkage to some remote station printer, computer server, or other information receiving system.

In particular, the subject concept is directed to a hydration assessment, potassium assessment, and serum pH assessment module which is incorporated within a system for measuring and predicting insulin dosing rates and for optimizing the assessed and predicted dosing rates.

Still further, the subject concept provides for a method and system for management of the blood dosing rate of a patient where calculations may be performed and displayed to aid the physician and/or caregiver in providing a proper insulin dosing rate to the patient.

Still further, the subject concept directs itself to a computer module which is coupled to an overall system and method for measuring and predicting insulin dosing rates which assesses the hydration state, potassium level, and serum pH blood value for use in managing hyperglycemia caused diabetic conditions.

### PRIOR ART

A number of prior art systems and methods have been available for measuring and predicting insulin dosing rates. In some of the prior art prediction and assessment systems, a simple equation of the form of blood glucose level of the patient minus a constant which remains fixed were multiplied by some other type of multiplier which was generally protocol dependent and based upon the input of the attending physician and/or caregiver. Such prior art methods produce predictions of future time interval blood glucose levels which were far out of the range of the patient's standard blood glucose reading.

In some prior art systems and methods, there was no ability to assess the hydration state, the potassium level, and the serum pH level of the patient to be used in conjunction with algorithms to determine proper management of hyperglycemic conditions.

In other prior art systems and methods, the attending physician and/or caregiver had provided for dosing rates which were based upon an initial time interval and did not take into account changes in the patient's physical parameters during the time interval leading to either an over-shoot or under-shoot of the blood glucose levels of the patient at the end of the time interval.

In other prior art systems and methods, there were no provisions made for addressing the patient's hydration state, potassium level, and/or serum pH value.

In other prior art systems, there was no provision for the portability of the overall system to allow the attending physician and/or caregiver the ability to permit movability from one patient to another.

In still other prior art systems relating to the prediction of blood glucose levels, there was no automatic system for transferring the patient's dosing rate data to an external device at a remote station.

### SUMMARY OF THE INVENTION

The subject concept is directed to a method for providing a diabetic keto-acidosis protocol for managing a patient's keto-acidosis state. The method includes the use of a computer processor for actuating a computer program to determine the patient's keto-acidosis state. Further, the method provides for inputting a multiplicity of patient parameters to the computer processor and resulting computer program where the patient parameters include the patient's blood glucose level, target patient blood glucose level, blood carbon dioxide level, sodium level, blood pH value, and the patient's potassium level.

A keto-acidosis protocol is initiated when the patient's blood glucose level is above a predetermined value. The patient's hydration state is assessed when the patient's blood carbon dioxide and venous pH value are above respective predetermined values and a displaying of any warning indicia and corrective action is provided.

The concept is further directed to a diabetic keto-acidosis protocol where it is assessed whether the patient is in hypovolemic shock, cardiogenic shock, or has mild hypotension, and the use of calculations based upon these characteristics to provide a proper administering of sodium chloride to the patient to bring the hydration state of the patient to a proper level.

Still further, the subject concept is directed to a system which assesses the potassium level of the patient and dependent upon the potassium level measured, directs the administration of insulin at a predetermined rate to the patient.

Still further, the subject concept directs itself to a further assessment of the patient's blood pH level and dependent upon the blood pH level of the patient, determines administration of sodium bicarbonate in predetermined amounts to aid and optimize the dosing rate of the insulin.

The subject system and method includes an iterative process where the patient's blood glucose level is measured at predetermined time intervals and calculations are made to recommend the dosing rates dependent upon the patient treatment.

The subject invention system and method is further directed to a blood-glucose monitoring system and keto-acidosis assessment mechanism whereby the attending physician and/or care provider is provided with alarms which may be visual and/or audio when the blood glucose level, potassium level, sodium level, or blood pH levels are external to some preferred target range.

### BRIEF DESCRIPTION OF THE DRAWINGS

FIG. 1 is a schematic diagram of an exemplary system configuration for measuring and predicting insulin dosing rates including a module directed to the diabetic keto-acidosis/hyperglycemia, hyperosmolarity module (DKA/HHS);

FIG. 2 is a computer flow diagram of an exemplary insulin dosing rate optimization program utilizing the DKA/HHS protocol module;

FIG. 3 is a computer flow diagram which provides for the overall block diagram for assessing hydration, assessing potassium, and assessing serum pH blood values of a patient;

FIG. 4 is a computer flow diagram detailing the hydration assessment of an individual patient;

FIG. 5 is a computer flow diagram detailing the assessment of potassium for an individual patient; and,

FIG. 6 is a computer flow diagram detailing the assessment protocol for assessing the blood pH level of an individual patient.

### DESCRIPTION OF THE PREFERRED EMBODIMENTS

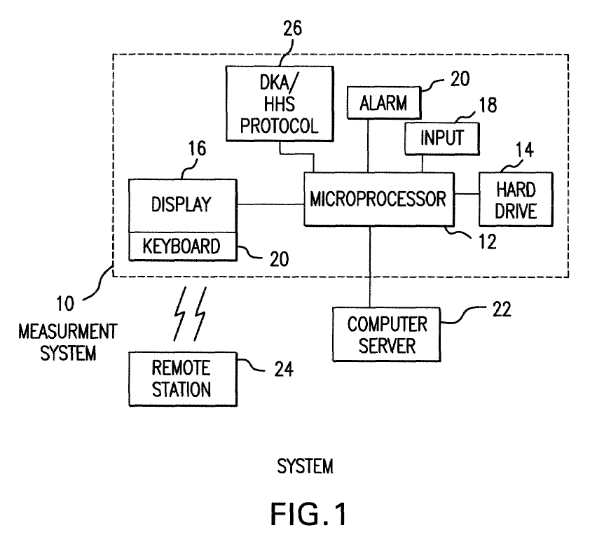

Referring now to FIG. 1, there is shown a schematic block diagram directed to an overall measurement and prediction system 10 for measuring and predicting an insulin dosing rate to bring a patient's blood glucose level into a preferred target range. Measurement and prediction system 10 may be a stand-alone system or may be portable, as will be described in following paragraphs.

Measurement system 10 includes microprocessor 12 for actuating a computer program having a plurality of modules for measurement and prediction of a patient's blood glucose levels and then further for storing and maintaining such patient's blood glucose levels taken at predetermined time intervals.

One module of microprocessor 12 is diabetic keto-acidosis/hyperglycemic/hyperosmolarity (DKA/HHS) 26 which forms a portion of the inventive concept of the subject invention.

Microprocessor 12 of prediction and measurement system 10 may be incorporated with hard drive 14 and includes display 16 which may be in the form of an LCD monitor or some other well-known type of commercially available display system. Display 16 may incorporate keyboard 20 or input data block 18. Alternatively, display 16 may be a touch screen type of input device which is well-known in the art and commercially available. Input 18 inputs among other parameters, a preferred target range for a particular patient's blood glucose level as well as inserting an initial blood glucose level, carbon dioxide level, sodium level, blood pH value, as well as a patient's potassium level at the initiation of the programs associated with microprocessor 12 and in particular, DKA/HHS 26.

Overall measurement and prediction system 10 further may include alarm mechanism 20 which would alert a user when the patient's glucose level is external to a preferred patient glucose target range, when the hydration level of a patient is external to standard hydration levels, when the patient's potassium level is greater than or less than a normal range, and when the patient blood serum pH value is further external an acceptable range.

All output from microprocessor 12 may be transmitted to remote station 24 either by shared wiring or through a wireless connection. Remote station 24 may be a remote printer, computer, computer server, or other peripheral device which is off-site with relation to measurement system 10.

Alarm 20 may be an audio alarm in the form of a buzzer or some like audio sounding mechanism, or may be a visual sensing system where the alarm warning is displayed on display 16.

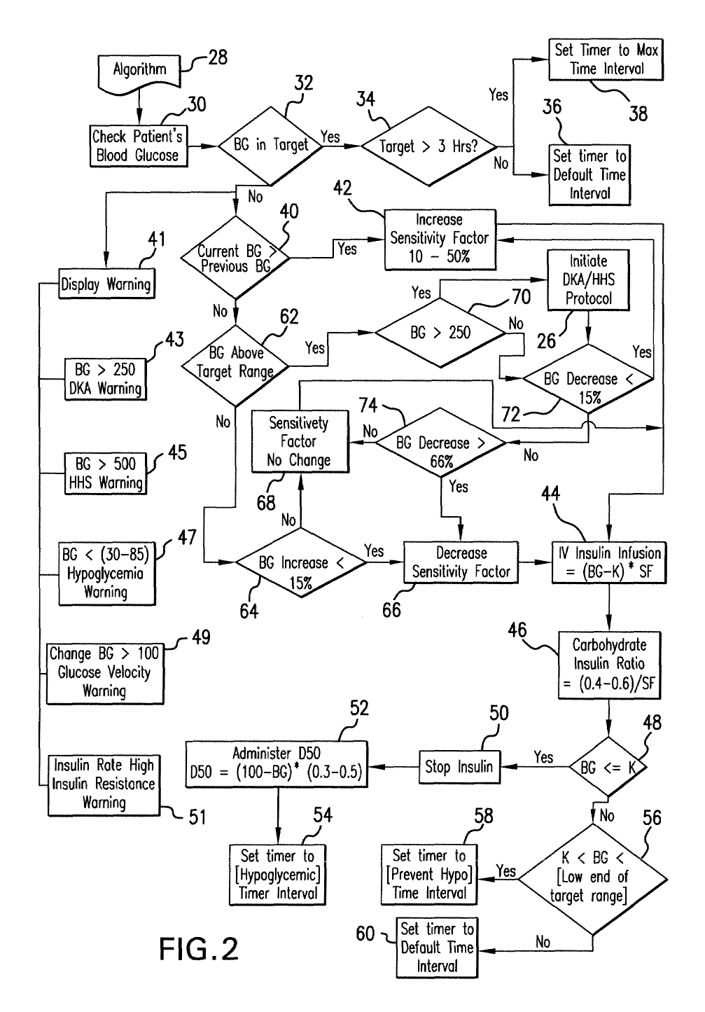

The subject invention concept is basically directed to DKA/HHS protocol block 26 shown in FIG. 2 in conjunction with algorithm 28 for determining various dosing rates associated with glucose levels of a patient. The patient's blood glucose level is inserted in block 30 of FIG. 2 for calculations to be made thereon. Logic within computer algorithm 28 passes the information to decision block 32 to determine whether the blood glucose level of the patient is within a predetermined target range. If the blood glucose level is found to be within the target range, the information passes to decision block 34 where it is determined whether the blood glucose level has been within the target range for more than a predetermined time interval (a common standard being three hours). If blood glucose level has not been within target range for a period greater than the predetermined time interval, then the physician or other caregiver sets a timer in block 36, where the timer is set to some default time interval, to provide a warning to actuate the system again. If the blood glucose level has been within the target range for more than the predetermined time interval, the logic then passes to block 38 where the timer is set to some maximum time interval empirically derived dependent upon the physician or attending caregiver's experience.

With relation to decision block 32, if the blood glucose level of the patient is not within the target range, information logic then passes to decision block 40 to determine whether the current blood glucose level is greater than the previously measured blood glucose level during a previous predetermined time.

Simultaneously, information flows from decision block 32 to display warning block 41 to provide a plurality of possible warning signals. Display warning 41 may include logic block 43 to provide a blood glucose warning that the blood glucose level is greater than 250 which is passed to display 16 for a visual warning to be seen, and/or to alarm 20 for issuance of a further sensory signal.

If the blood glucose level is found to be greater than 500, a hyperglycemic/hyperosmolarity warning is provided in block 45. Both the blood glucose warning block 43 and the hyperglycemic/hyperosmolarity warning 45 is particularly used in conjunction with the DKA/HHS logic block 26.

Further with regard to the warning block's warning system, when the blood glucose level is less than 30-85, a hyperglycemia warning may be provided in block 47. A change of blood glucose level greater than 100 warning is provided in block 49 and an insulin rate high insulin resistance warning in block 51. All of these warnings may be provided on display 16 with an associated alarm 20 being actuated. Further, the information may be sent to remote station 24 external the overall measurement and prediction system 10.

Returning now to FIG. 2, when the blood glucose is found not to be within the target range in decision block 32, information passes to previously described decision block 40 where it is determined whether the current blood glucose level is greater than the previous blood glucose level.

If the current blood glucose level is greater than the previous blood glucose level in block 40, information passes to logic block 42 where the sensitivity factor is increased between 10%-50% (the actual percentage being protocol dependent).

Once the sensitivity factor is increased in logic block 42 by a predetermined amount (either based upon the physician or caregiver's empirical input or further protocol), the information then flows to block 44 where the dosing rate or IV insulin infusion rate is calculated.

The calculation is then made in accordance with the following formula:

DR=(BG−K)·SF (1)

where:
DR=dosing/infusion rate
BG=patient blood glucose level
K=constant where 40<K<80

and:
65<K<80 for capillary measurement
40<K<70 for arterial measurement
40<K<70 for venous measurement
65<K<84 interstitial measurement

The constant K may also vary on rate of blood glucose change and target blood sugar ranges, however, such is maintained in accordance with the criteria of 40<K<80.

Logic flow then goes from block 44 to block 46 where the carbohydrate insulin ratio is calculated in general as being 0.4-0.6 divided by the sensitivity factor. The carbohydrate insulin ratio is a number used to calculate how much insulin is needed to offset carbohydrate intake in order that a patient's blood glucose value is not affected.

Subsequent to the carbohydrate insulin ratio being determined in block 46, information logic then flows to block 48 which is a decision block where a determination is made as to whether the blood glucose is less than or equal to K.

If the blood glucose in decision block 48 is less than K, then logic flows to block 50 where insulin dosing is terminated. Once the insulin dosing is terminated in block 50, the logic flows to block 52 for administering D50 which is a dextrose 50% solution in accordance with the formula amount of D50−(100−BG)×0.4. However, administration may be between the range of 0.3-0.5 dependent upon the protocol set up by the physician an/or caregiver. The timer is then set to the hypoglycemic time interval in block 54 where the hypoglycemic time interval is reset to a predetermined time interval which may be at any time interval decided by the physician or caregiver, but is generally in the range of 30 minutes.

Returning to decision block 48, in the event that the blood glucose level is greater than K, the logic flows to block 56 which is a further decision block where it is determined whether the blood glucose level is within the range of K<BG<(low end of target range). If the BG is within the range, then the logic flows to block 58 where the timer is then set to a time interval for prevention of hyperglycemia. If the BG is not within the range of K and the lower end of the target range, then the logic flows to block 60 for setting of the timer to a default time interval.

Returning now to decision block 40 where it is determined that the current BG level is less than the previous blood glucose level, the logic flows to block 62 which is a decision block and a determination is made if the blood glucose level is above the target range. If the blood glucose level is not above the target range, then the logic flows to block 64 which is a decision block to determine whether the blood glucose increase is less than 15%. If the blood glucose level in decision block 64 is determined to be less than 15%, then information logic flows to block 66 where the sensitivity factor is decreased in accordance with the protocol of the physician or caregiver. Subsequently responsive to the decreased sensitivity factor, the logic then flows into previously described logic block 44.

If the blood increase is less than 15% in decision block 64, information passes to logic block 68 where there is no sensitivity factor change and the flow of data then passes directly back to logic block 44 to determine the insulin infusion rate.

If the blood glucose is found to be above the target range in decision block 62, the information flow then passes to decision block 70 where it is determined whether the blood glucose level is greater than 250. If the blood glucose is calculated to be greater than 250, logic then flows to DKA/HHS block 26 for initiating the diabetic keto-acidosis/hyperglycemia/hyperosmolarity protocol.

Prior to the detailed discussion associated with the initiation of the DKA/HHS protocol, a brief description of the case where the blood glucose is less than or equal to 250 found in block 70 follows. In the event that the blood glucose level is equal to or less than 250, then the information flows to block 72 which is a decision block to determine whether the blood glucose level decrease is less than 15%. If the blood glucose level is found to be less than 15% in decision block 72, information then passes back to information block 42 for increasing the sensitivity factor and then passage of the information to block 44. In the event that the blood glucose decrease is greater than 15%, the logic then passes to decision block 74 where it is determined whether the blood glucose decrease is greater than 66%. If it is greater than 66%, then logic flows to logic block 66 for the decrease of the sensitivity factor. If the blood decrease is less than 66%, then the logic flows from decision block 74 to block 68 where there is no change in the sensitivity factor and then the information flows to insulin infusion block 44, as previously described.

Referring now to decision block 70 where it is determined that the blood glucose level is greater than 250, DKA/HHS protocol block 26 is initiated and is detailed in following paragraphs.

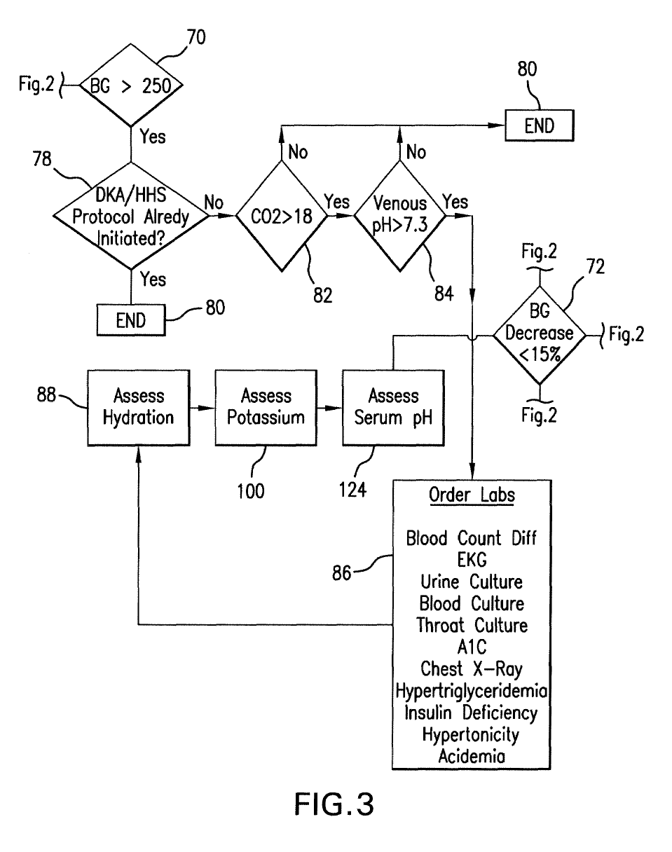

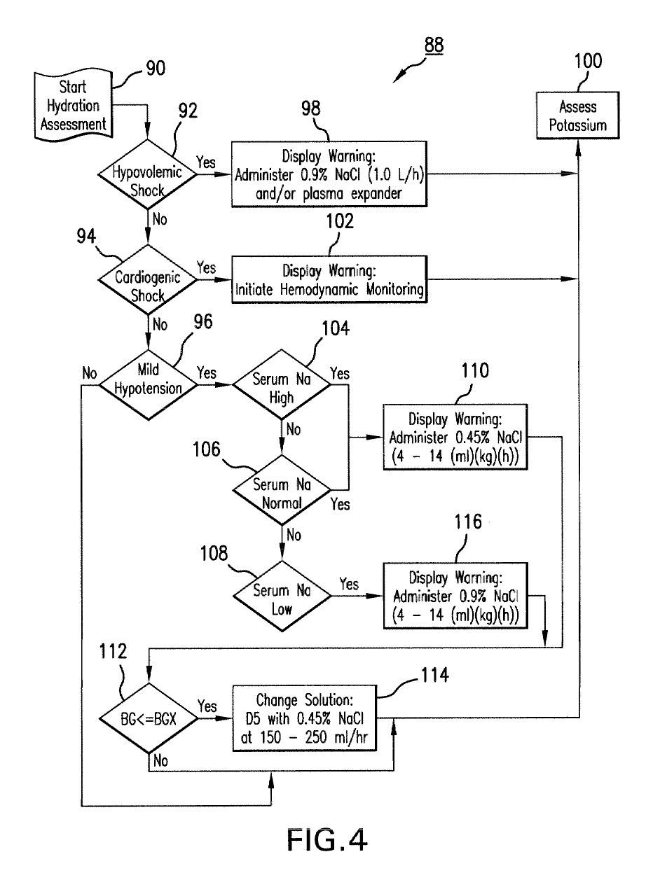

Referring now to FIG. 3, subsequent to the BG being determined to be greater than 250 in block 70, information passes to block 78 which is a decision block to determine whether the DKA/HHS protocol has previously been initiated. If the protocol had been previously initiated, the program then moves to block 80 which is an end program block for termination of the program procedure. If the DKA/HHS protocol has not been initiated in decision block 78, the information is directed to decision block 82 where the carbon dioxide level of the patient is determined. If the carbon dioxide level is greater than 18 mmHg, the logic passes to decision block 84 where the pH is assessed. If the venous pH is greater than 7.3, information moves to logic block 86. In the event that the carbon dioxide is less than or equal to 18, or the venous pH is less than or equal to 7.3, logic then passes to end program block 80.

Where the carbon dioxide level is greater than 18 mmHg and the venous pH is greater than 7.3, the information as previously stated goes to information block 86 where lab work is ordered by the physician or caregiver. Such lab work in block 86 may have been previously obtained and includes a number of well-known laboratory orders. Once the input has been obtained from block 86, information passes to assess hydration block 88 for assessing the hydration level of the patient. The hydration assessment logic block 88 is shown in FIG. 4.

Upon hydration assessment being initiated in block 90, information then moves to decision block 92 where it is determined whether the patient is in hypovolemic shock, whether the patient is in cardiogenic shock in decision block 94, or has mild hypotension in decision block 96. The decision of decision blocks 92, 94, and 96 may be empirically derived by the physician or caregiver or alternatively in protocol form from various information inserted from the lab information block 86.

If the patient is determined to be in hypovolemic shock in decision block 92, information passes to information block 98 where a warning is provided and may be displayed on display 16 and/or through alarm 20. At this point, there is the administration of approximately 0.9% sodium chloride at approximately 1 liter per hour and/or the administration of a plasma expander. Information then passes to assess potassium block 100, as shown in FIGS. 3 and 4.

If the patient is determined not to be in hypovolemic shock in decision block 92, information passes to decision block 94 where it is determined whether the patient is in cardiogenic shock. If the patient is in cardiogenic shock, once again, a warning is provided on display 16 and/or alarm 20 and there is an initiation of hemodynamic monitoring as provided in block 102. Once again, the information then passes to assess potassium block 100 shown in both FIGS. 3 and 4.

Assuming that the patient has not been determined to be in hypovolemic shock in block 92 and cardiogenic shock in block 94, information then passes to information block 96 to determine whether the patient is in mild hypertension. If the patient is not in mild hypertension, information then flows to assess potassium block 100.

In the event that the patient is in mild hypertension, information then passes to decision blocks 104, 106, and 108 where a decision is made as to whether the serum sodium is high, normal, or low. If the serum sodium is high, information flows to warning information block 110 where the display warning can be provided on display 16 or through alarm 20 and there is the administration of a 0.5% sodium chloride between 4-14 mL(kg)h)). Information from logic block 110 then passes to logic block 112 where a decision is made as to whether the blood glucose is less than or equal to the target blood glucose range. If the blood glucose level is less than or equal to the target range blood glucose level, the information passes to information block 114 for a change in the solution to D5 (5% dextrose) with 0.45% sodium chloride at between 150-250 mL/hr. Once the change in solution has been made, the information then passes to the next block for assessing potassium in logic block 100.

If the serum sodium is not found to be high or normal, information passes from decision blocks 104 and 106 to 108 where the logic flows to information block 116 where the warning is displayed once again on the display 16 and/or through the alarm 20 and there is the administration of 0.9% sodium chloride solution (between 4-14 ml(kg)(h)). As previously noted, once the information passes from block 116, the logic flows to previously described decision block 112 to determine whether the blood glucose level is less than or equal to the blood glucose target range. Depending upon the value of the blood glucose, the information then passes to the previously described logic block 114 or in the alternative, passes to the assess potassium block 100.

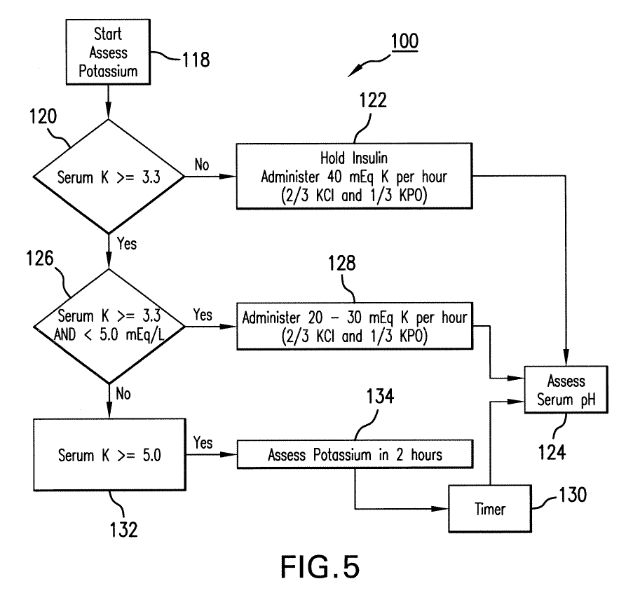

Referring now to FIG. 5, such details the logic flow in assess potassium block 100. FIG. 5 shows the initiation of the logic block 100 in block 118. In formation then flows to decision block 120 where it is determined whether the serum potassium is less than 3.3 mEq/L (milli equivalents per liter). If the serum potassium is less than 3.3 mEq/L, then information passes to block 122 where insulin dosage is terminated and there is the administering of potassium in the amount of 40 mEq per hour. Subsequently, the information then passes to block 124 which is assessing the serum pH block shown in FIG. 5 and FIG. 3.

In the event that the serum potassium is equal to or greater than 3.3 as found in decision block 120, information passes to information block 126 where it is determined whether the serum potassium is within the range of 3.3 and 5.0 mEq/L. If the serum potassium is within the specified range, then information passes to block 128 where administration of 20-30 mEq is provided per hour to the patient. Information may then pass to a timer 130 for giving a warning signal within some predetermined time interval. Simultaneously, the information further passes to assess serum pH block 124.

If it is found that the serum potassium is not within the specified range in decision block 126, the information passes to block 132 and then directly to an assess potassium block 134 where the information is passed to timer 130 for providing a warning signal within a predetermined time such as two hours.

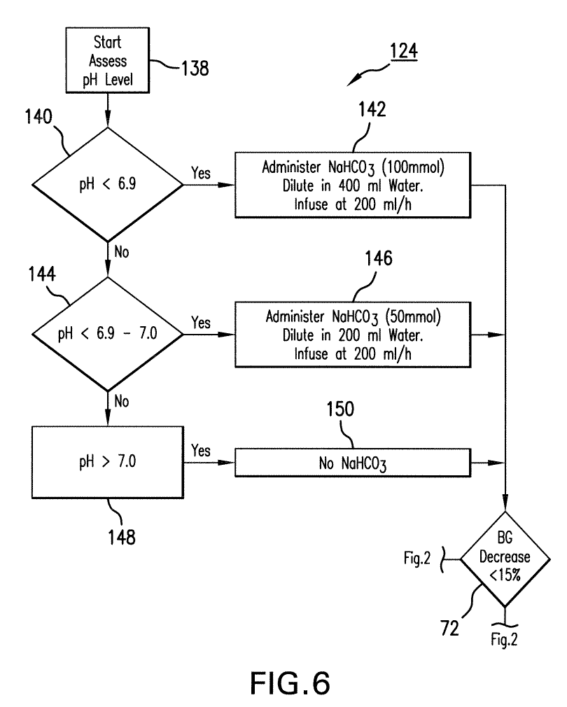

Returning now to FIG. 3, once the hydration assessment has been made in block 88, the potassium assessment being made in block 100, information then flows to assess serum pH block 124 shown in FIG. 6.

Information begins with start pH level block 138 and passes to decision block 140 where it is determined whether the pH level is less than 6.9. If the pH level is less than 6.9, information passes to information block 142 for administering sodium bicarbonate (NaHCO₃) in a predetermined amount and then the information flows to decision block 72 of FIG. 2 which has previously been described.

If the pH level is equal to or greater than 6.9, then the information logic passes to decision block 144 to determine whether the pH level is within the range of 6.9-7.0. If the pH level is within the range 6.9-7.0, then the information is directed to block 146 where there is a further administration of sodium bicarbonate in a predetermined amount as required by the physician or caregiver. If the pH value is greater than 7.0, information passes to information block 148 and then to block 150 where there is no sodium bicarbonate being provided to the patient. Information once again as previously described for decision blocks 140 and 144 passes to decision block 72 of FIG. 2.

Once completed in FIG. 3, the total hydration assessment, potassium assessment, and serum pH values have been calculated and assessed in information blocks 88, 100, 124. Finally, output in FIG. 3 from serum pH assessment is sent to decision block 72 of FIG. 2 for further calculations in accordance with the previous descriptions made.

It is appreciated by those skilled in the art that changes may be made to the embodiments described above without departing from the broad inventive concept thereof. It is understood, therefore, that this invention is not limited to the particular embodiments disclosed, but is provided to cover modifications within the spirit and scope of the present invention, as defined by the appended Claims.

What is claimed is:

1. A method for providing a diabetic keto-acidosis protocol for managing a patient's keto-acidosis state including the steps of:
(a) establishing a computer processor for actuating a computer program for determining the patient's keto-acidosis state;
(b) inputting a plurality of patient parameters to said computer processor, said patient parameters including a patient's blood glucose level, a target patient blood glucose level, a patient's blood carbon dioxide level, a patient's blood sodium level, a patient's blood pH value, and a patient's blood potassium level;
(c) initiating a keto-acidosis protocol when said patient's blood glucose level is above a predetermined value;
(d) assessing a patient's hydration state when said patient's blood carbon dioxide and blood pH value are above respective predetermined values; and
(e) displaying a hydration warning indicia and hydration corrective action indicia to be taken on a display monitor when said patient's hydration state is assessed.

2. The method as recited in claim 1, where the steps of assessing said patient's hydration state includes the steps of determining whether a patient is in hypovolemic shock, cardiogenic shock or has mild hypotension and displaying respective warning indicia on said display monitor when said hypovolemic shock, cardiogenic shock or mild hypotension is determined.

3. The method as recited in claim 2, where the steps of determining a patient's mild hypotension state includes the steps of assessing said patient's blood sodium level and displaying a hypotension warning indicia on said display monitor responsive to said patient's sodium level.

4. The method as recited in claim 3, where the steps of assessing said patient's sodium level includes the steps of:
(a) displaying a first hypotension warning indicia on said display monitor when said patient's blood sodium level is above approximately 4 mg/dL;
(b) displaying a second hypotension warning indicia on said display monitor when said patient's blood sodium level is less than approximately 4 mg/dL; and
(c) displaying corrective action indicia on said display monitor responsive to said first hypotension warning indicia and said second hypotension warning indicia.

5. The method as recited in claim 4, where the step of assessing said patient's blood sodium level is followed by the step of determining whether said patient's blood glucose level is less than said patient's target blood glucose level.

6. The method as recited in claim 5, where the step of determining said patient's blood sodium level includes the steps of administering a 5% dextrose solution with 0.45% sodium chloride at a predetermined rate when said patient's blood glucose level is less than said patient's target blood glucose level.

7. The method as recited in claim 1, where the step of assessing said patient's hydration state is followed by the steps of assessing said patient's blood potassium level and displaying a respective potassium warning indicia responsive to said patient's blood potassium level.

8. The method as recited in claim 7, where the step of determining said patient's blood potassium level includes the steps of:
(a) displaying a first potassium warning indicia on said display monitor when said patient's blood potassium level is less than 3.3 mEq/L;
(b) displaying a second potassium warning indicia on said display monitor when said patient potassium level is greater than 3.3 mEq/L and less than 5.0 mEq/L;
(c) displaying a third potassium warning indicia on said display monitor when said patient's blood potassium level is greater than 5.0 mEq/L; and
(d) displaying a potassium corrective action indicia on said display monitor responsive to said first, second and third potassium warning indicia.

9. The method recited in claim 8 where the step of displaying said third potassium warning indicia includes the step of repeating step (c) after a predetermined time interval.

10. The method as recited in claim 7, where the step of determining said patient's blood potassium level is followed by the steps of assessing said patient's blood pH value and displaying a respective blood pH value warning indicia responsive to said patient's blood pH value.

11. The method as recited in claim 10, where the step of assessing said patient's blood pH value includes the step of comparing said patient's blood pH value to a plurality of blood pH values.

12. The method as recited in claim 11 where the step of comparing said patient's blood pH value includes the steps of:
(a) displaying a first blood pH value warning indicia on said display monitor when said patient's blood pH value is less than 6.9;
(b) displaying a second blood pH value warning indicia on said display monitor when said patient's blood pH value is greater than 6.9 and less than 7.0;
(c) displaying a third blood pH value warning indicia on said display monitor when said patient's blood pH value is greater than 7.0; and,
(d) displaying a blood pH value corrective action indicia on said display monitor responsive to said first, second and third blood pH value warning indicia.

13. A system for providing a diabetic keto-acidosis protocol comprising:
(a) a computer processor having patient parameter data input thereto, said patient parameter data being input to said computer processor including a patient's blood glucose level, a target patient blood glucose level, a patient's blood carbon dioxide level, a patient's blood sodium level, a patient's blood pH value, and a patient's blood potassium level;
(b) a patient hydration computer program for assessing a patient's hydration state;
(c) a patient potassium computer program for assessing said patient's blood potassium level;
(d) a patient blood pH value computer program for assessing said patient's blood pH value;
(e) a display monitor coupled to said computer processor for displaying predetermined indicia responsive to actuation of said patient hydration computer program, said patient potassium computer program and said patient blood pH value computer program.

14. The system as recited in claim 13, where said patient hydration computer program includes means for determining whether a patient is in hypovolemic shock, cardiogenic shock or has mild hypotension, said means for determining being displayed on said display monitor as hydration warning indicia and hydration corrective action indicia.

15. The system as recited in claim 14 where said patient hydration computer program includes means for assessing said patient's blood sodium level within said means for determining mild hypotension and a hypotension warning indicia on said display monitor responsive to said patient's blood sodium level.

16. The system as recited in claim 15, where said hypotension warning indicia includes:
(a) a first hypotension warning indicia on said display monitor when said patient's blood sodium level is above approximately 4 mg/dL;
(b) a second hypotension warning indicia on said display monitor when said patient's blood sodium level is less than approximately 4 mg/dL; and
(c) a corrective action indicia on said display monitor responsive to said first and second hypotension warning indicia.

17. The system as recited in claim 13 where said patient potassium computer program includes means for assessing said patient's blood potassium level and a potassium warning indicia for being displayed on said display monitor responsive to said patient's blood potassium level.

18. The system as recited in claim 17, where said potassium warning indicia includes:
(a) a first potassium warning indicia on said display monitor when said patient's blood potassium level is less than 3.3 mEq/L;
(b) a second potassium warning indicia on said display monitor when said patient's blood potassium level is greater than 3.3 mEq/L and less than 5.0 mEq/L;
(c) a third potassium warning indicia on said display monitor when said patient's blood potassium level is greater than 5.0 mEq/L; and
(d) a potassium corrective action indicia on said display monitor responsive to said first, second and third potassium warning indicia.

19. The system as recited in claim 13, where said patient blood pH value computer program includes means for assessing said patient's blood pH value and a blood pH value warning indicia for being displayed on said display monitor responsive to said patient's blood pH value.

20. The system as recited in claim 19, where said blood pH value warning indicia includes:
(a) a first blood pH value warning indicia on said display monitor when said patient's blood pH value is less than 6.9;
(b) a second blood pH value warning indicia on said display monitor when said patient's blood pH value is greater than 6.9 and less than 7.0;
(c) a third blood pH value warning indicia on said display monitor when said patient's blood pH value is greater than 7.0; and,
(d) a blood pH value corrective action indicia on said display monitor responsive to said first, second and third blood pH value warning indicia.
